(19) United States
(12) Patent Application Publication
(10) Pub. No.: US 2018/0036561 A1
(43) Pub. Date: Feb. 8, 2018

(54) PORTABLE CHEMICAL OXYGEN GENERATOR

(71) Applicant: THE REGENTS OF THE UNIVERSITY OF MICHIGAN, Ann Arbor, MI (US)

(72) Inventors: Kevin R. Ward, Superior Township, MI (US); Gary S. Huvard, Richmond, VA (US); Patrick M. Huvard, New Kent, VA (US)

(21) Appl. No.: 15/556,376

(22) PCT Filed: Mar. 10, 2016
(86) PCT No.: PCT/US16/21718

§ 371 Date: Sep. 7, 2017

Related U.S. Application Data
(60) Provisional application No. 62/132,840, filed on Mar. 13, 2015.

(51) Int. Cl. A62B 7/08; C01B 13/02; A61M 16/10

(52) U.S. Cl. CPC A62B 7/08; A61M 16/101; C01B 13/0207

(57) ABSTRACT

The disclosure provides oxygen generating compositions including sodium percarbonate, manganese dioxide, and trisodium phosphate dodecahydrate which generates high purity breathable oxygen. The disclosure further provides methods of generating oxygen and portable chemical oxygen generators including the oxygen generating compositions of the disclosure.

### STATEMENT OF US GOVERNMENT SUPPORT

This invention was made with government support under N00014-07-1-0526 awarded by the Office of Naval Research. The government has certain rights in the invention.

### FIELD OF THE DISCLOSURE

This disclosure relates generally to oxygen generating compositions, methods of producing oxygen, and portable chemical oxygen generators. More particularly, this disclosure relates to oxygen generating compositions that can be stored long-term and do not produce toxic by-products or volatile organic compounds, thereby providing high purity breathable oxygen.

### BACKGROUND

A chemical oxygen generator is a device that releases oxygen via a chemical reaction. Chemical oxygen generators are important for providing emergency oxygen in situations in which other methods like electrolysis or oxygen tanks are not feasible. Chemical oxygen generators are currently used in aircraft, submarines, and by firefighters and mine rescue crews, as well as a backup supply for the International Space Station. Other uses can be anywhere a compact oxygen generator with a long shelf life is needed, as in automated external defibrillator locations, military operations, and third-world clinics.

U.S. Pat. No. 7,171,964 teaches an oxygen generation system which includes a reaction chamber that is partially filled with hydrogen peroxide of 7.5% in an aqueous solution. The portable oxygen generation system further includes a second chamber that is adjacent to the first chamber containing manganese dioxide powder acting as the catalyst. By breaking a seal between the two chambers, the hydrogen peroxide solution mixes with the manganese dioxide to produce oxygen which is released through an exit tube. This chemical reaction is highly exothermic. It produces 23.4 kcal per mole of hydrogen peroxide (1938 cal/L O₂ produced). Due to the large amount of hydrogen peroxide that might be present at the start of the reaction, the temperature in the reaction chamber will rapidly rise causing the aqueous solution of hydrogen peroxide to boil. The high reaction temperature typically requires some form of insulation in order for the system to be handled by a user safely.

In order to reduce the safety risk associated with aqueous solutions of hydrogen peroxide, solid hydrogen peroxide adducts such as sodium percarbonate and urea hydrogen peroxide have been used in chemical oxygen generator systems. U.S. Pat. No. 5,823,181 describes a system including a chemical module mounted in the reaction chamber containing a hydrogen peroxide and catalyzer. A plunger mounted on the reaction chamber to break the chemical module, enabling the two chemicals to mix with water react and produce oxygen. Because of the large amount of oxygen produced, the chemical reaction is highly exothermic.

Various designs have been sought to protect the user from the heat generated by the exothermic reaction. Heat exchangers are often impractical and costly in portable units. In U.S. Pat. No. 7,407,632, for instance, the reaction chamber is enclosed in an exterior housing creating an “air insulator” to prevent the heat from reaching the outer surface of the generator and allow safe handling. Such design does not prevent the reaction chamber from reaching high temperature.

Other designs seek to control the temperature inside the reaction chamber by capturing part of the heat generated in the reaction. U.S. Pat. No. 4,548,730 teaches how heat-absorbing hydrated salts can be intermixed with oxygen materials to absorb excessive heat released upon exothermic chemical decomposition of hydrogen peroxide. A limitation with such hydrated salts is that they release carbon dioxide upon heating.

Similarly, U.S. Pat. No. 8,147,760 teaches an oxygen generation system in which oxygen is generated chemically from the catalytic decomposition of hydrogen peroxide by a catalyst such as manganese dioxide to yield water and oxygen. Hydrogen peroxide is produced from the slow decoupling of urea hydrogen peroxide. The urea hydrogen peroxide is formulated into controlled release tablets which dissolve slowly in water and release hydrogen peroxide. The aqueous solution is nearly saturated with urea in order to prevent the rapid dissolution of solid urea in the device, used as a cooling agent, when the device is activated. The dissolution of urea is endothermic and too rapid a cooling at the start of the reaction quenches the reaction and slows the generation of oxygen to unacceptably low rates. The solvent comprises a mixture of water and polyethylene glycol (PEG). PEG is a chemically inert, water miscible non-solvent for urea and urea hydrogen peroxide. The presence of PEG decreases the mass of urea required to pre-saturate the mixture and limits the rate of dissolution of urea hydrogen peroxide, thereby aiding control over the rate of oxygen release by the device. However, urea rapidly hydrolyzes to toxic ammonia at temperatures above 50° C. The rate of urea hydrolysis increases dramatically with temperature which limits the maximum temperature allowed for the solution to about 50° C. as the oxygen-producing reaction proceeds. Furthermore, the hydrolysis reaction proceeds even at room temperature, and the slow generation of ammonia in the water-PEG-urea solution limits the expected shelf life of the device to about six months. After which time, the smell of ammonia from the solution is noticeable.

There is therefore a need for an efficient, portable oxygen generating composition and oxygen generation system that can be stored long term, produce a predictable and constant flow of oxygen for an extended period of time, and provide a safe delivery of oxygen to the user.

### SUMMARY

One aspect of the disclosure provides an oxygen generating composition including sodium percarbonate, manganese dioxide, and trisodium phosphate dodecahydrate. The composition can further include water.

Another aspect of the disclosure provides a method of generating oxygen including contacting an oxygen generating composition of the disclosure with water.

Another aspect of the disclosure provides a portable chemical oxygen generator including a housing enclosing a reaction chamber including a first compartment containing an oxygen generating composition of the disclosure and a second compartment containing water, and an activation means attached to the housing for opening the sealed second compartment and for contacting the composition with the water.

A still further aspect of the disclosure provides a portable chemical oxygen generator including a housing enclosing a reaction chamber comprising a first compartment containing an oxygen generating composition of the disclosure and an activation means attached to the housing for providing water and contacting the composition with the water.

Further aspects and advantages will be apparent to those of ordinary skill in the art form a review of the following detailed description. While the compositions, methods, and devices are susceptible of embodiments in various forms, the description hereafter includes specific embodiments with the understanding that the disclosure is illustrative, and is not intended to limit the invention to the specific embodiments described herein.

### DESCRIPTION OF THE DRAWINGS

FIG. 1 is a photograph of a mixture of sodium percarbonate with manganese dioxide and trisodium phosphate dodecahydrate that has been held in an oven for 11 months at 50° C.

FIG. 2 is a phase diagram of sodium percarbonate.

FIG. 3 is a plot of the fraction of O₂ released over time.

FIG. 4 is a plot of the rate of oxygen produced over time.

FIG. 5 is a plot of the temperature inside a reaction chamber over time.

### DETAILED DESCRIPTION

This disclosure relates to chemical oxygen generating compositions. Particularly, it relates to compositions that can be stored long-term, exhibit controlled heat production, and do not produce toxic by-products or volatile organic compounds, which allow these compositions to thereby generate high-purity, breathable oxygen. Further, the disclosure relates to methods of producing oxygen and portable chemical oxygen generators.

One aspect of the disclosure provides an oxygen generating composition including sodium percarbonate, manganese dioxide, and trisodium phosphate dodecahydrate. In some embodiments, the composition can further include water. In various cases, the composition can be substantially free of polyethylene glycol. Optionally, the composition can consist essentially of sodium percarbonate, manganese dioxide, and trisodium phosphate dodecahydrate. In various cases, oxygen the generating compositions of the disclosure can be stored indefinitely (e.g., at least 1 day, at least 1 week, at least 1 month, up to 1 month, at least 2 months, at least 3 months, at least 4 months, at least 5 months, at least 6 months, at least 7 months, at least 8 months, at least 9 months, at least 10 months, at least 11 months, at least 12 months, etc.) at temperatures of at least 50° C., and the compositions produce substantially no toxic by-products or volatile organic compounds, thereby generating high purity breathable oxygen.

Another aspect of the disclosure provides a method of generating high purity breathable oxygen including contacting an oxygen generating composition of the disclosure with water. The amount of oxygen generating composition and water can be selected such that oxygen is generated at a rate of at least 1 L/min, at least 2 L/min, at least 3 L/min, at least 4 L/min, or at least 6 L/min for 20 minutes. Further, the oxygen generating composition of the disclosure can be prepared such that one or more of foam, toxic by-products, and volatile organic compounds are minimal, or not produced, when the composition is contacted with the water.

Another aspect of the disclosure provides a portable chemical oxygen generator including a housing enclosing a reaction chamber including a first compartment containing an oxygen generating composition of the disclosure and a second sealed compartment containing water, and an activation means attached to the housing for opening the sealed second compartment and for contacting the composition with the water. The amount of oxygen generating composition enclosed in the first compartment and the amount of water contained in the second sealed compartment can be selected such that oxygen is generated at a rate of at least 1 L/min, at least 2 L/min, at least 3 L/min, at least 4 L/min, or at least 6 L/min for 20 minutes. The oxygen generating composition can be prepared such that high purity breathable oxygen is generated and one or more of foam, toxic by-products, and volatile organic compounds are minimized or not produced when the composition is contacted with the water.

A still further aspect of the disclosure provides a portable chemical oxygen generator including a housing enclosing a reaction chamber comprising a first compartment containing an oxygen generating composition of the disclosure and an activation means attached to the housing for providing water and contacting the composition with the water. Advantageously, the water can be provided at the point of use. The amount of oxygen generating composition enclosed in the first compartment and the amount of water contacted with the composition can be selected such that oxygen is generated at a rate of at least 1L/min, at least 2 L/min, at least 3 L/min, at least 4 L/min, or at least 6 L/min for 20 minutes. Further, the oxygen generating composition can be prepared such that high purity breathable oxygen is generated and one or more of foam, toxic by-products, and volatile organic compounds are minimized or not produced when the composition is contacted with the water.

The use of urea hydrogen peroxide (UHP) is avoided for the presently disclosed compositions, which has previously been used in oxygen generators, but exhibits various undesired properties as discussed above. Urea hydrogen peroxide is thermally unstable and somewhat hygroscopic, properties that make urea hydrogen peroxide unsuitable if the chemical oxygen generating device is to be stored and used at elevated temperatures (e.g., greater than about 80° F. (about 27° C.)). Thus, urea hydrogen peroxide-based devices are generally unsuitable for use by the military, by third-world clinics, or generally in any environment where temperature cannot be strictly controlled. Thus, provided herein are compositions which do not comprise urea hydrogen peroxide.

Further, because mixtures of urea hydrogen peroxide and manganese dioxide are unstable, the manganese dioxide must be kept separate from the urea hydrogen peroxide during transport and storage. Even in powder form at room temperature, trace amounts of hydrogen peroxide exist on the surface of the powder particles. This hydrogen peroxide reacts with the manganese dioxide to release oxygen and water. The released water decomposes more UHP, releasing more hydrogen peroxide. The process continues autocatalytically and within a short amount of time (e.g., an hour or so) the UHP is visually mushy and obviously decomposing. Thus, the manganese dioxide catalyst must be separated from the UHP until the oxygen generating reaction is desired. It has been found that because of this separation, UHP-based devices often fail to reach the desired oxygen generation rate. Manganese dioxide slurries are not physically stable, as the magnesium dioxide readily settles out of the suspension or slurry, due to the much greater density of manganese dioxide compared to water. Once settled, it is difficult to uniformly re-disperse the manganese dioxide. It was found that if the manganese dioxide is not fully dispersed when the aqueous phase is contacted with the UHP, the UHP does not provide the optimal rate of oxygen generation, because the effective concentration of manganese dioxide catalyst is much lower than the concentration required to generate oxygen at the desired rate.

Yet another disadvantage of a UHP-based oxygen generation composition is that urea is a by-product of UHP decomposition or is used as a cooling agent to combat the effects of the exothermic reaction. However, urea is hydrolytically unstable at temperatures above 50° C. and forms ammonia which is toxic, even at part per million levels. Thus, ammonia can form not only during the exothermic oxygen production, but also when the device is stored at elevated temperatures. Accordingly, the temperature of a UHP-based device must always remain below 50° C. The use of urea as a cooling agent is also problematic because it has been found that unless the dissolution of urea is controlled, urea can substantially lower the temperature of the reactor contents almost immediately upon contact of the water, thereby quenching the hydrogen peroxide decomposition reaction. As such, the oxygen generating compositions disclosed herein do not use urea and do not generate urea or ammonia, thereby avoiding the dangers of the presence of urea.

Ranges may be expressed herein as from “about” or “approximately” one particular value and/or to “about” or “approximately” another particular value. When such a range is expressed, another contemplated embodiment includes from the one particular value and/or to the other particular value. Similarly, when particular values are expressed as approximations, but use antecedents such as “about,” “at least,” “at most,” “less than about” or “more than about” it will be understood that the particular value forms another embodiment.

As used herein and unless specified otherwise, the terms “wt %” and wt. %” are intended to refer to the composition of the identified element in parts by weight of the entire composition.

As used herein, the term “substantially free of” indicates that the identified element or feature is not present in the composition, or if it is present in the composition, is present in an amount of less than about 1.0 wt %. In some cases, the identified element or feature is present in an amount of less than about 0.5 wt %, less than 0.4 wt %, less than 0.3 wt %, less than 0.2 wt %, or less than 0.1 wt %. With respect to the amount of hydrogen peroxide vapor present in an oxygen stream, “substantially free of” indicates that the hydrogen peroxide vapor is not present in the oxygen stream or is present in an amount of less than the short term exposure limit. For example, an oxygen stream that is substantially free of hydrogen peroxide vapor has less than 3 ppm, less than 2 ppm, less than 1 ppm, or less than 0.5 ppm hydrogen peroxide.

### Oxygen Generating Compositions

The oxygen generating compositions of the disclosure comprise sodium percarbonate. Sodium percarbonate (NaPerc) is an adduct of sodium carbonate and hydrogen peroxide, having an empirical formula Na₂CO₃.1.4H₂O₂. The NaPerc adduct is a granular powder that decomposes in water to release hydrogen peroxide. NaPerc is stable at temperatures of greater than 50° C. and far less hygroscopic than other hydrogen peroxide adducts, such as UHP. In some embodiments, the NaPerc is an uncoated pure grade of NaPerc. Many of the coatings used to protect NaPerc from water vapor during storage can contribute to foam formation. Thus, in some cases, the NaPerc of the composition is uncoated. Suitable uncoated, pure grade NaPerc is available from Solvay Chemicals under the trade name FB®400. Further, in some cases the NaPerc is substantially free of foam-producing additives.

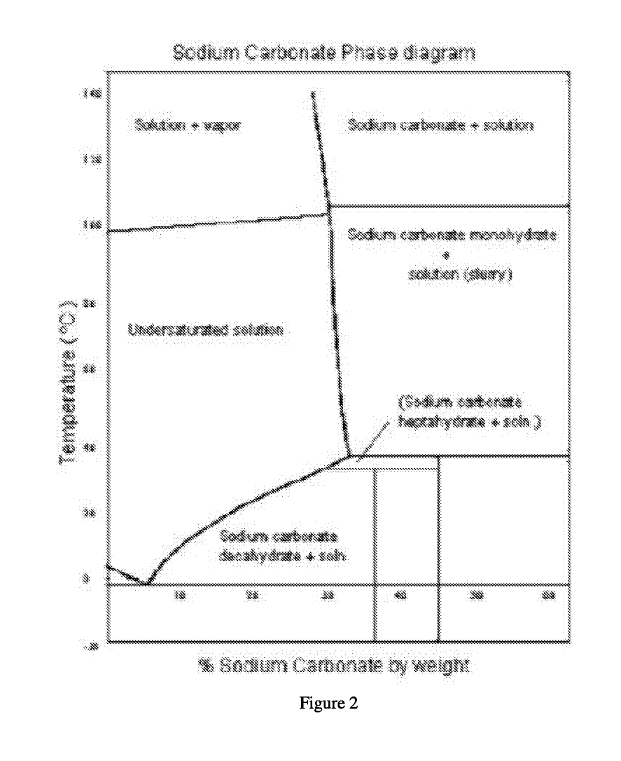

Multiple variables affect the oxygen release rate: the temperature of the solution, the rate of dissolution of hydrogen peroxide and the amount of catalyst dispersed in the solution. If too large a quantity of hydrogen peroxide adduct is dissolved at once in the aqueous solution, the rate of oxygen production will increase rapidly and uncontrollably. NaPerc adduct addresses these concerns, as it is highly stable and dissolves slowly in water thereby slowly releasing hydrogen peroxide and sodium carbonate which, below about 40° C., forms the decahydrate in aqueous solution. If the solution is maintained at a temperature at or below about 40° C., saturation of the solution by sodium carbonate decahydrate can limit the continued dissociation of the adduct. The solubility of sodium carbonate is affected by the temperature of the water. As shown in FIG. 2, at above about 40° C., the conversion of the sodium carbonate decahydrate to the heptahydrate and monohydrate forms drives the continued dissociation of sodium percarbonate to hydrogen peroxide. Therefore, the continued dissociation of the adduct and release of oxygen at the desired rate can be achieved by controlling the temperature of the aqueous solution. Because of the slow dissolution of NaPerc in water, the oxygen production by an oxygen generating composition of the disclosure is adiabatic. Therefore, when an oxygen generating composition of the disclosure is used in a portable chemical oxygen generator, no heat is transferred from the portable chemical oxygen generator to the surroundings.

The NaPerc can be included in the composition in an amount effective to produce a desired volume of O₂ at a desired rate (e.g., at least 1 L/min, at least 2 L/min, at least 3 L/min, at least 4 L/min, or at least 6 L/min for 20 minutes). One of ordinary skill in the art will readily appreciate that the amount of oxygen produced will depend upon the amount of sodium percarbonate used, the amount of water used and the temperature of the water, as the oxygen is produced from the decomposition of hydrogen peroxide which is liberated from the sodium percarbonate in the presence of water. For example, if 2000 g of water are used, about 500 g of sodium carbonate (corresponding to about 740 g NaPerc) will dissolve at room temperature and about 660 g of sodium carbonate (about 997 g NaPerc) will dissolve at 40° C. Without intending to be bound by theory, it is believed that above 40° C., the continued dissolution of NaPerc may be driven by the conversion of sodium carbonate heptahydrate to the more soluble monohydrate form. As shown in FIG. 2, at above about 40° C., the sodium carbonate decahydrate form converts to the heptahydrate and monohydrate forms. As demonstrated in the examples below, about 1200 g NaPerc can be used to produce about 114 L (STP) of O₂.

NaPerc can be present in the composition in an amount in a range of about 10 wt % to about 80 wt %, about 20 wt % to about 80 wt %, about 30 wt % to about 80 wt %, about 40 wt % to about 80 wt %, about 50 wt % to about 80 wt %, 50 wt % to about 75 wt %, about 60 wt % to about 75 wt %, about 65 wt. % to about 75 wt %, or about 70 wt % to about 75 wt % based on the total weight of the NaPerc, manganese dioxide, and trisodium phosphate dodecahydrate.

The hydrogen peroxide generated from the NaPerc decomposes to water and oxygen in the presence of a peroxide decomposition catalyst. The oxygen generating compositions of the disclosure comprise manganese dioxide (MnO₂) as the peroxide decomposition catalyst. The catalyst can be present in the form of a powder, e.g. a fine powder. MnO₂ can be obtained in various powder sizes ranging from about 5 micron diameter to more than 500 micron diameter. Further, MnO₂ can be activated to provide a better oxidation catalyst. As used herein, activated MnO₂refers to MnO₂ subjected to a series of heat/oxygen/inert gas treatments that are used by those skilled in the art to produce a MnO2 powder that is especially active as a catalyst for chemical oxidation reactions. One of ordinary skill in the art will appreciate that each form of MnO₂ will provide a different reaction rate when used in the same weight percent in the compositions of the disclosure. One of ordinary skill in the art will further appreciate that the settling rate of larger diameter MnO₂ particles limits the practicality of using such larger diameter particles in the compositions of the disclosure. In some embodiments, the MnO₂ has an average diameter of about 5 micron to 75 micron, about 5 micron to about 70 micron, about 5 micron to 65 micron, about 5 micron to about 60 micron, about 5 micron to about 55 micron, about 5 micron to about 50 micron, about 10 micron to about 50 micron, about 15 micron to about 50 micron, about 20 micron to about 50 micron, about 25 micron to about 50 micron, about 30 micron to about 50 micron, about 35 micron to about 50 micron, about 40 micron to about 50 micron, for example about 40 micron, about 41 micron, about 42 micron, about 43 micron, about 44 micron, about 45 micron, about 46 micron, about 47 micron, about 48 micron, or about 50 micron. It has been found that when the MnO₂ particles are of about 50 micron, dispersion of the particles in the aqueous suspension is facilitated by the rising bubbles of oxygen as it is formed. In some embodiments the MnO₂ has a particle size in a range of about 10 micron to about 50 micron and is not activated. In some embodiments, the MnO₂ has a particles size of about 5 micron to about 10 micron and is activated.

The MnO₂ can be included in the composition in an amount effective to catalyze the decomposition of hydrogen peroxide and produce the desired volume of O₂. As demonstrated in the examples below, about 3.5 g MnO₂ particles having a mesh size of 325 (about 44 micron) can be used to produce about 114 L (STP) of O₂.

The amount of MnO₂ used in the composition depends on the mesh size of the MnO₂ and on the degree of activation of the MnO₂ for use as a chemical oxidant. Such activated MnO₂ powders are also very active in the decomposition of hydrogen peroxide and while activated MnO₂ powders can be used in the compositions of the disclosure, the use of activated MnO₂ is not required. Whether activated or not activated, the amount of MnO₂ required is typically about 5% or less of the weight of NaPerc in the composition, for example up to about 5%, up to about 4%, up to about 3%, up to about 2%, up to about 1%, up to about 0.9%, up to about 0.8%,up to about 0.7%, up to about 0.6%, up to about 0.5%, up to about 0.4%, up to about 0.3%, up to about 0.2%, up to about 0.1%, up to about 0.05%, up to about 0.01%, up to about 0.005%, up to about 0.004%, up to about 0.003%, up to about 0.002%, or up to about 0.001% of the weight of NaPerc in the composition. As demonstrated in the examples below, an unactivated MnO₂ having a mesh sieve of 325 (about 44 micron) was used at a level of 0.3% of the weight of NaPerc in the composition.

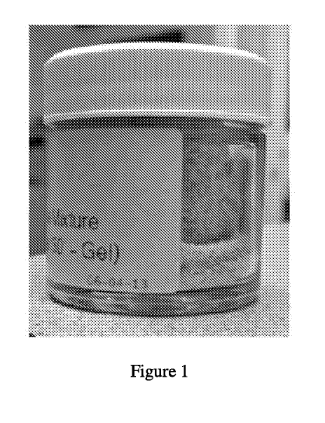

NaPerc is stable even in contact with unactivated MnO₂, as long as the NaPerc is maintained in a substantially dry condition. Thus, the two materials can be mixed together without visually observable or significant NaPerc decomposition, even at temperatures of 50° C. or higher. Without intending to be bound by theory, it is believed that NaPerc is stable enough that trace amounts of hydrogen peroxide do not form on the surface of the powder and thus no reaction takes place until water is introduced, dissolving the NaPerc and liberating hydrogen peroxide which can then react with the catalyst. As shown below in the examples, FIG. 1 demonstrates that there is no detectable decomposition of NaPerc when stored in the presence of MnO₂ in an oven at 50° C. for at least one year.

The oxygen generating compositions of the disclosure further comprises a cooling agent, such as trisodium phosphate dodecahydrate, sodium tetraborate decahydrate, disodium phosphate heptahydrate, disodium phosphate dodecahydrate, and combinations of the foregoing. In some embodiments, the cooling agent is selected from the group consisting of trisodium phosphate dodecahydrate, disodium phosphate heptahydrate, disodium phosphate dodecahydrate, and combinations thereof. Trisodium phosphate dodecahydrate (TSP) is white solid having the formula Na₂PO₄.12H₂O. TSP can be used in an oxygen generating composition as a cooling agent as it dissolves endothermically, having a cooling capacity of 40.25 cal/g. Further, it has been found that it is possible to contact TSP with water without generating an unacceptably large endotherm. As such, contacting TSP with water will not quench the decomposition of hydrogen peroxide and does not waste the cooling capacity of TSP upon initial contact with water. TSP has a more limited solubility in water than other cooling agents, such as urea, and while the solubility of THP increase as the temperature of the water rises, the solubility of THP increases more slowly than the solubility of urea increases. Further, TSP lowers the surface tension of sodium carbonate solutions, thereby inhibiting or minimizing the formation of foam.

The rate of dissolution of TSP was also found to be affected by the form of TSP. The reaction profile of TSP powder and small TSP tablets were different and thus TSP can be used as a powder, a tablet, or combinations thereof, and the form can be selected based on the desired reaction profile. For example, TSP powder, given its much higher surface area, dissolves more quickly than the TSP tablets. In some embodiments, TSP is used as a tablet, either alone or in combination with TSP powder. In embodiments wherein the TSP is used as a tablet, the tablets are about 0.25 inch to about 0.38 inch (about 0.63 cm to about 0.97 cm) in diameter and about 0.12 inches to about 0.19 inches (0.31 cm to about 0.50 cm) in thickness. The tablets may have flat profiles on each face or they may have slightly outwardly curved faces. In embodiments wherein TSP is used as a combination of powder and tablets, the ratio of powder to tablets can be in a range of about 30% powder/70% tablet to about 70% powder/30% tablet.

The inclusion of the cooling agent affects the temperature of the reaction and thus, the rate of release of O₂. For example, TSP can regulate the temperature profile of the reaction to result in a substantially constant rate of oxygen productions. As the NaPerc is consumed, the temperature of the reaction increases in order to achieve the constant release rate at the desired rate of oxygen production. One of ordinary skill in the art will readily appreciate that the desired dissolution rate of TSP is achieved by selecting a TSP powder to tablet ratio for a given amount of MnO₂. The cooling agent can be present in the composition in an amount in a range of about 20 wt % to about 90 wt %, about 20 wt % to about 80 wt %, about 20 wt % to about 70 wt %, about 20 wt % to about 60 wt %, about 50 wt %, or about 20 wt % to about 40 wt %, or about 20 wt % to about 30 wt % based on the total weight of the NaPerc, MnO₂, and TSP. For example, TSP can suitably be included in an amount in a range of about 325 g to about 815 g.

In various cases, the oxygen generating compositions disclosed herein have a ratio of NaPerc:MnO₂:TSP of about 20:0.001:79.999 to about 30:1:69. For example, the ratio of these components in a composition disclosed herein can be 21:0.001:78.999, 22:0.001:77.999; 23:0.001:76.999, 24:0.001:75.999, 25:0.001:74.999, 26:0.001:73.999, 27:0.001:72.999, 28:0.001:71.999. 29:0.001:70.999, 30:0.001:69.999, 21:0.002:78.998, 22:0.002:77.998; 23:0.002:76.998, 24:0.002:75.998, 25:0.002:74.998, 26:0.002:73.998, 27:0.002:72.998, 28:0.002:71.998. 29:0.002:70.998, 30:0.002:69.998, 21:0.01:78.99, 22:0.01:77.99; 23:0.01:76.99, 24:0.01:75.99, 25:0.01:74.99, 26:0.01:73.99, 27:0.01:72.99, 28:0.01:71.99. 29:0.01:70.99, 30:0.01:69.99, 21:0.05:78.95, 22:0.05:77.95; 23:0.05:76.95, 24:0.05:75.95, 25:0.05:74.95, 26:0.05:73.95, 27:0.05:72.95, 28:0.05:71.95. 29:0.05:70.95, 30:0.05:69.95, 21:0.1:78.9, 22:0.1:77.9; 23:0.1:76.9, 24:0.1:75.9, 25:0.1:74.9, 26:0.1:73.9, 27:0.1:72.9, 28:0.1:71.9. 29:0.1:70.9, 30:0.1:69.9, 21:0.3:78.7, 22:0.3:77.7; 23:0.3:76.7, 24:0.3:75.7, 25:0.3:74.7, 26:0.3:73.7, 27:0.3:72.7, 28:0.3:71.7. 29:0.3:70.7, or 30:0.3:69.7.

In some embodiments, the oxygen generating composition further includes water. In such embodiments, the oxygen generating composition does not produce toxic by-products or volatile organic compounds. Unlike similar UHP-based compositions that must remain below 50° C. so as to not produce toxic urea, the NaPerc-based compositions disclosed herein comprise inorganic compounds only, and, therefore, cannot generate volatile organic compounds. Nor do the compositions disclosed herein generate poisonous ammonia gas. Accordingly, the NaPerc-based compositions provide high-purity, breathable oxygen without needing to be maintained below 50° C., and can also operate at higher temperatures, for example, at least 80° C., or at least 90° C., or about 80 to about 90° C.

Further, because the NaPerc-based compositions can be operated at temperatures higher than 50° C., the water does not need to be mixed with other solvents to control the rate at which the NaPerc dissolves. Unlike UHP-based compositions which utilize a water/polyethylene glycol (PEG) mixture to control the dissolution rate of UHP, the compositions disclosed herein do not require PEG to control the dissolution rate of NaPerc in water. Water/PEG/urea solutions utilized in UHP-based compositions are required to be partially saturated with urea in order to prevent substantial cooling of the reaction mixture upon contact of the UHP-containing solids and the water/PEG/urea solution. For the NaPerc containing compositions disclosed herein, water alone is needed to contact the NaPerc containing solids to begin generation of oxygen. Furthermore, water/PEG/urea mixtures slowly produce toxic ammonia upon storage at room temperature. Ammonia cannot be produced by the compounds utilized in the NaPerc compositions disclosed herein, an important advantage over UHP-containing compositions.

The disclosure further provides a method of generating oxygen including contacting an oxygen generating composition of the disclosure with water. As used herein, “contacting” can include any of flowing the water past/through the oxygen generating composition or immersing the oxygen generating composition in the water. In some embodiments, the contacting will occur by opening a valve between an upper compartment containing water and a lower compartment containing the oxygen generating composition and allowing gravity to drain the water into the lower compartment to initiate the reaction and release of oxygen. The relative amounts of the sodium percarbonate, manganese dioxide, and trisodium phosphate dodecahydrate used in the method of generating oxygen can be any amounts disclosed herein for the oxygen generating composition.

On contact with the water, the NaPerc adduct decomposes to produce sodium carbonate and hydrogen peroxide. The hydrogen peroxide further decomposes to water and oxygen upon contact with the MnO₂ catalyst. The decomposition rate of hydrogen peroxide depends on the concentration of the hydrogen peroxide and on the reaction temperature. In various embodiments, the manganese dioxide is present in an excess to overwhelm the hydrogen peroxide with an overabundance of catalyst. In some embodiments, an amount of MnO₂ provided in the range of about 0.3% to about 1% of the weight of NaPerc is sufficient to react the H₂O₂ produced at a substantially instantaneous rate such that H₂O₂ is decomposed to oxygen as quickly as the H₂O₂ is released from the decomposition of NaPerc. In such embodiments, the rate of production of oxygen is substantially equivalent to the rate of decomposition of the NaPerc and the concentration of H₂O₂ in the aqueous phase of the reaction remains at all times at exceedingly low concentrations. For this reason, the exiting oxygen stream is also substantially free of H₂O₂.

The water is also used as a heat sink to absorb some of the heat produced by the oxygen generation reaction. The enthalpy of the decomposition reaction of hydrogen peroxide is 46.8 kcal per mole of oxygen produced or 1.95 kcal per liter of oxygen produced at 20° C. In a system designed to generate 6 liters of oxygen per minute, the system will produce 11.7 kcal per minute. A system producing 6 liters of oxygen per minute and activated at ambient temperature of 20° C., then, would bring one liter of water to a boil in less than seven minutes. A method of producing oxygen strictly using water as a heat sink would therefore require large amounts of water, significantly adding to the weight of the apparatus or device in which the method is carried out. In contrast, the methods disclosed herein comprise an oxygen generating composition including TSP as a cooling agent. The endothermic dissolution of TSP can absorb all or part of the excess energy produced during the exothermic chemical decomposition of hydrogen peroxide, thereby requiring less water than if water alone were the heat sink.

The oxygen generating composition and the water can be provided in any amounts suitable for initiating and maintaining the oxygen generation reaction. Suitable weight ratios of water to NaPerc can include weight ratios in a range of about 1.5:1 to about 2.5:1, for example about 1.7:1. One of ordinary skill in the art will readily appreciate that when the amount of water is too low relative to the NaPerc, the rate of oxygen production will be too rapid and the temperature of the composition will increase. One of ordinary skill in the art will also readily appreciate that when the amount of water added is above the disclosed range, the water will add unnecessary mass and the rate of oxygen generation will not be constant throughout the lifetime of the NaPerc. The weight ratios of NaPerc to water can be selected such that oxygen is generated at a rate of at least 1 L/min, at least 2 L/min, at least 3 L/min, at least 4 L/min, or at least 6 L/min for 20 min. For example, in some embodiments, the composition comprises 70 wt % to 75 wt % sodium percarbonate, 0.1 wt % to 0.3 wt % 44 μm manganese dioxide, and 20 wt % to 30 wt % trisodium phosphate dodecahydrate and oxygen is generated at a rate of at least 6 L/min for about 20 minutes.

### Portable Chemical Oxygen Generators

The disclosure further provides a portable chemical oxygen generator including a housing enclosing a reaction chamber including a first compartment containing an oxygen generating composition as disclosed herein and a second sealed compartment containing water, and an activation means attached to the housing for opening the sealed second compartment and for contacting the composition with the water.

The reaction chamber that is enclosed by the housing can be made of any suitable materials capable of containing the reaction materials inside the reaction chamber while allowing the produced oxygen through. Suitable materials can include a dual membrane filter. An example of a dual membrane filter is disclosed in U.S. Pat. No. 8,417,760, which is hereby incorporated by reference in its entirety. The dual membrane filter disclosed in U.S. Pat. No. 8,417,760 is a filter having a first membrane made from a hydrophobic material that is resistant to liquids and solids, but permeable to gasses, such as a high-density polyethylene material such as Tyvek®. Tyvek® is a commercially available nonwoven fabric from Dupont (Wilmington, Del.) that has moderately high gas permeability and is also highly water resistant. Gas permeability is measured as the airflow in mL/min through a 10 cm² area according to ISO-5636-3 (Bendsten Air Permeability). A high density polyethylene material such as Tyvek® 1073 or Tyvek® 1059 has a Bendsten Air Permeability of approximately 60 mL/min/cm². In order to handle an oxygen flow of 6,000 mL/min, there is a need for a membrane surface area of at least 100 cm² or 16 in². Water resistance (the “hydrostatic head” or “hydrohead”) is defined as the height of water column in centimeters that the membrane can withstand without leaking according to DIN EIN 20811. If 3 L of a solution were contacted with a membrane having a surface area of 100 cm², the liquid covering the membrane would be 30 cm deep. Thus, a membrane having a hydrohead in excess of 30 cm would not allow the liquid to leak through. Tyvek® materials typically exhibit hydroheads in excess of 140 cm. Therefore, a first membrane made from such material would allow oxygen gas and water vapor to pass through but would keep the solid and liquid reactants contained in the reaction chamber. The second membrane of the dual membrane filter disclosed in U.S. Pat. No. 8,417,760 comprises a super-absorbent material and is located between the first membrane and the housing. By super-absorbent material is meant a hydrophilic material which absorbs and retains aqueous solutions and which, in deionized and distilled water, can absorb up to 800 times its dry weight. The super-absorbent fabric absorbs both water vapor and any unreacted hydrogen peroxide vapor and water vapor and allows high purity oxygen gas to flow through. The second membrane can be made from a composite of melt blown polypropylene fibers combined with super-absorbing polyacrylamide fibers such as that produced by Evolution Sorbent Products (Chicago, Ill.).

The second sealed compartment can be made of any suitable watertight material. An activation mechanism ruptures the sealed watertight compartment releasing the water. The water then contacts the oxygen generating composition. The activation mechanism may comprise a cord or safety pin attached to the side of the sealed watertight compartment which cord or safety pin releases the contents of the sealed watertight compartment when pulled. In some embodiments, the user may physically squeeze the sealed watertight chamber in order to activate the mechanism by breaking a frangible seal. Alternatively, the activation mechanism may comprise a handle, knob, or screw which when turned opens a valve or punctures the watertight compartment releasing the contents of the sealed watertight compartment into the first compartment.

Alternatively, the device can be manually activated by a user who simply pours the designated amount of water from a suitable container into the chemical-containing chamber through a replaceable cap or plug. Alternative embodiments include a two compartment device wherein the water is provided in a lower chamber and the oxygen generating composition of the disclosure is provided in an upper chamber, the chambers being connected through a connection having an open and closed position. In such embodiments, the device would be activated by turning the device upside down such that the water chamber becomes the upper chamber, and moving the connection into the open position in order to drain the water into oxygen generating composition chamber. The connection can be opened, for example, by twisting one or both of the chambers at right angles (or through 180°) to the other. Those skilled in the art can appreciate there are many possible options for contacting the water with the oxygen generating composition.

The disclosure further provides a portable chemical oxygen generator including a housing enclosing a reaction chamber comprising a first compartment containing an oxygen generating composition of the disclosure and an activation means attached to the housing for providing water and contacting the composition with the water. The reaction chamber that is enclosed by the housing can be made of any suitable materials capable of containing the reaction materials inside the reaction chamber while allowing produced oxygen through. Suitable materials can include a dual membrane filter as described previously.

The activation means attached to the housing can be any means that allow water to be added to the housing and reaction chamber. For example, the activation means can be a sealable watertight second compartment connected to the first compartment by a sealed channel and further having a sealable opening through which water can be introduced to the sealable watertight second compartment. Once the water is added to the watertight second compartment and the compartment sealed, the sealed channel can be opened allowing the water to flow into first compartment. Optionally, the channel can be re-sealed after the water enters the first compartment such that the generated oxygen does not escape from the first compartment into the second compartment. Advantageously, water can be added at the site of use and need not be transported and/or stored in the reaction chamber of a portable chemical oxygen generator. One skilled in the art can appreciate that there are many different arrangements for contacting water with the oxygen generating composition.

The portable chemical oxygen generators of any aspect of the disclosure can further include a means of removing residual hydrogen peroxide from the oxygen prior to the delivery to a user. Hydrogen peroxide, like water, exerts a finite vapor pressure over the solution and may therefore be present at a low concentration in the oxygen that forms. Inhalation of hydrogen peroxide can cause irritation to the lungs. The means of removing residual hydrogen peroxide can be any means suitable, for example providing a membrane capable of chemically decomposing any residual hydrogen peroxide. For example, membranes capable of chemically decomposing any residual hydrogen peroxide include superabsorbent membranes, that absorb water vapor and any hydrogen peroxide vapor, comprising small particles of MnO₂ such as the membranes disclosed in U.S. Pat. No. 8,147,760, herein incorporated by reference in its entirety. Notwithstanding the contemplation of potential hydrogen peroxide residuals in the oxygen stream produced by the chemical oxygen device disclosed herein, no such hydrogen peroxide residuals have been measured or detected in any of the laboratory experiments carried out during the development of the oxygen generating compositions of the disclosure.

Specific contemplated aspects of the disclosure herein are described in the following numbered paragraphs.
1. An oxygen generating composition comprising
   sodium percarbonate;
   manganese dioxide; and
   a cooling agent selected from the group consisting of trisodium phosphate dodecahydrate, disodium phosphate heptahydrate, disodium phosphate dodecahydrate, and combinations thereof.
2. The composition of paragraph 1, wherein the composition is substantially free of polyethylene glycol.
3. The composition of paragraph 1 or 2, wherein the composition is a powder mixture.
4. The composition of any one of paragraphs 1 to 3, wherein the cooling agent comprises trisodium dodecahydrate.
5. The composition of any one of paragraphs 1 to 4, wherein the cooling agent is a powder, a tablet, or a combination thereof.
6. The composition of any one of paragraphs 1 to 5, wherein the sodium percarbonate is present in the composition in an amount in a range of about 10 wt % to about 80 wt %.
7. The composition of any one of the paragraphs 1 to 6, wherein the manganese dioxide is present in the composition in an amount in a range of about 0.001 wt % to about 5.0 wt %, based on the weight of NaPerc.
8. The composition of any one of paragraphs 1 to 7, wherein the cooling agent is present in the composition in an amount in a range of about 20 wt % to about 90 wt %.
9. The composition of any one of paragraphs 1 to 8, wherein the sodium percarbonate is uncoated and substantially free of foam-producing additives.
10. The composition of any one of paragraphs 1 to 9, consisting essentially of sodium percarbonate;
   manganese dioxide; and
   trisodium phosphate dodecahydrate.
11. The composition of any one of paragraphs 1 to 10, wherein the composition, when stored at a temperature of about 50° C. for at least 1 year, retains at least 99% of the initial amount of sodium percarbonate.
12. The composition of any one of paragraphs 1 to 11, further comprising water.
13. The composition of paragraph 12, wherein the composition has a water to sodium percarbonate ratio in a range of about 1.5:1 to about 2.5:1, or about 1.7:1.
14. The composition of paragraph 12 or paragraph 13, wherein the water is substantially free of polyethylene glycol.
15. A method of generating oxygen comprising
   contacting the composition of any one of paragraphs 1 to 11 with water to generate oxygen.
16. The method of paragraph 15, wherein the oxygen is generated at a rate of at least 1 L/min, at least 2 L/min, at least 3 L/min, at least 4 L/min, or at least 6 L/min.
17. The method of paragraph 15 or paragraph 16, wherein the composition comprises 70 wt % to 75 wt % sodium percarbonate, 0.1 wt % to 0.3 wt % manganese dioxide, and 20 wt % to 30 wt % trisodium phosphate dodecahydrate and oxygen is generated at a rate of at least 6 L/min for 20 minutes.
18. The method of any one of paragraphs 15 to 17, wherein the oxygen generation does not produce foam.
19. The method of any one of paragraphs 15 to 18, wherein the generation of oxygen is adiabatic.
20. A portable chemical oxygen generator comprising
   a housing enclosing a reaction chamber comprising a first compartment containing the composition of any one of paragraphs 1 to 11 and a second sealed compartment containing water; and
   an activation means attached to the housing for opening the sealed second compartment and for contacting the composition with the water.
21. A portable chemical oxygen generator comprising
   a housing enclosing a reaction chamber comprising a first compartment containing the composition of any one of paragraphs 1 to 11; and
   an activation means attached to the housing for providing water and contacting the composition with the water.
22. The portable chemical oxygen generator of paragraph 20 or paragraph 21, wherein the water is substantially free of polyethylene glycol.
23. The portable chemical oxygen generator of paragraph 22, wherein each of the composition and water is substantially free of polyethylene glycol.

The above described aspects and embodiments can be better understood in light of the following examples, which are merely intended to illustrate the compositions, methods and devices, and are note meant to limit the scope thereof in any way.

### EXAMPLES

Oxygen production rates were measured gravimetrically in all examples. Specifically, the entire oxygen-generating device was mounted on a digital balance and the weight of the device was recorded with time after the addition of water to establish an initial total weight prior to the onset of the reaction and the loss of weight resulting from oxygen flow out of the device. Gravimetric measurement is an absolute means of establishing the loss rate of oxygen and is preferred over all flowmeter type measurement devices. Such flowmeter type devices typically require periodic calibration and, in almost all cases, such calibrations are carried out using gravimetric measurement procedures.

### Example 1

The stability of a NaPerc, MnO₂, and TSP mixture was investigated as follows. 10.0 g NaPerc, 0.1 g of 44 micron MnO₂, and 1.5 g of TSP were added to a jar in powder form and powders were mixed thoroughly by using a laboratory spatula followed by shaking the jar violently. Another jar was prepared in the same way, except a silica gel pack containing 5 grams of silica gel (Uline Corporation) was added to the jar after the powders were mixed. The jars were covered with slightly loosened caps and placed in a 50° C. oven. The jars were allowed to remain in the oven for more than 1 year. At no time during storage under these conditions was there any measureable weight loss, and there was no visually detectable decomposition of the NaPerc. Visual inspection of the jar without the silica gel pack detected some clumping of the three components after several months but the clumps broke up easily again on shaking the jar. The clumps continued to form thereafter and were periodically broken up by shaking. The jar with the silica gel pack did not develop any clumps at any time and the powder remained loose and free-flowing throughout the time in the oven. FIG. 1 shows a photograph of the mixture of NaPerc, MnO₂, and TSP powder that was stored with a silica gel pack. If visually detectable decomposition of the NaPerc had occurred, the mass in the jar would have been fused together in one or more hard clumps which would not breakable by shaking the jar. A similar experiment using urea hydrogen peroxide, even at room temperature, lead to a wet, mushy mixture within several hours.

Thus, Example 1 demonstrates that oxygen generating compositions according to the disclosure are stable at 50° C. for at least 1 year.

### Example 2

The production of oxygen by a NaPerc, MnO₂, and TSP mixture was investigated as follows. 1205 g NaPerc (Solvay FB 400, 13.5% active O₂), 3.50 g MnO₂ (unactivated, 44 micron, Sigma Aldrich), and 125 g of TSP powder (Fisher Scientific) and 300 g of TSP tablets (with 5 wt % kaolin as a tableting aid, 10 mm diameter, 3 mm thick, flat faces)were added to a well-insulated, adiabatic reaction chamber.

The reaction chamber comprised a metal cylinder of 12″ in diameter and 5″ height. A metal top added another 0.5″ to the overall height. A 1″ diameter, capped pipe was attached in the center of the metal top and a barbed fitting was attached to the top about 3″ from the center as an outlet for the oxygen. The capped pipe was used as the addition port for water. The entire assembly was covered with a 1-1.5″ thick layer of closed cell, polyurethane insulating foam of about 3 lb/ft³ density. A similar foam piece was fabricated to fit over the top during an experiment. When assembled, the reactor was fully insulated on bottom, sides, and top.

To carry out the experiment, the cylindrical vessel was loaded with the desired amounts of NaPerc, TSP powder, TSP tablets, and MnO₂. The top and insulation were added and the pipe cap was temporarily removed for the addition of 2 L of water. The cap was tightened on the pipe and oxygen allowed to flow from the barbed fitting. The entire assembly was placed on a digital balance and the mass was recorded every minute until the end of the reaction. The mass lost by the vessel was due to the loss of oxygen and a small, almost negligible, amount of water vapor.

Thus, Example 2 demonstrates the production of oxygen by an oxygen generating composition of the disclosure.

### Example 3

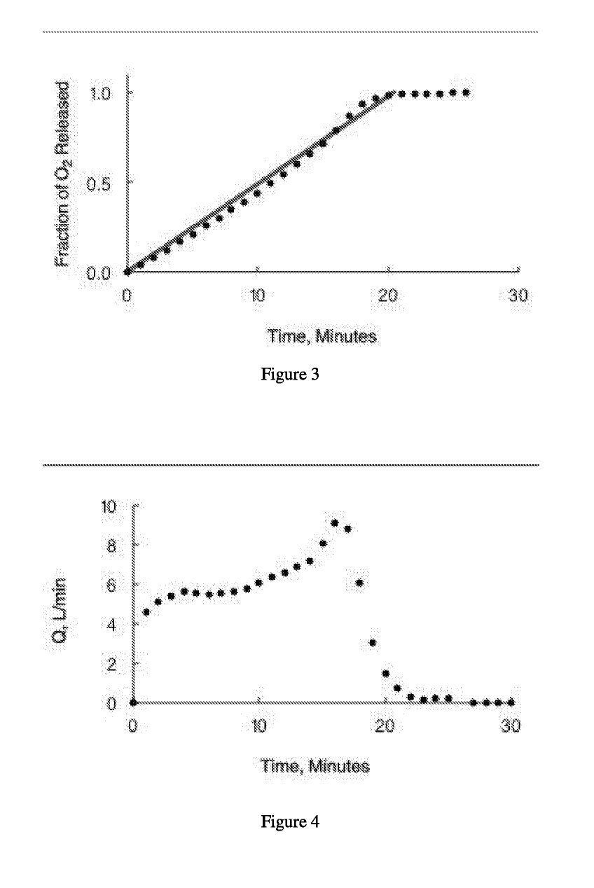

The loss of oxygen, as recorded by the mass lost from the reaction chamber of Example 2, was graphed as in FIG. 3. The theoretical yield of O₂ produced from 1205 g NaPerc having 13.5% active O2 is 162.67 g O₂ when all NaPerc has decomposed and all H₂O₂ has reacted. The fraction of theoretical oxygen produced is therefore the mass lost from the reaction chamber divided by 162.67. This value is also called the “fractional conversion” at any given time during the reaction. The fractional conversion vs. time is shown in FIG. 3.

The total weight loss of the device after a completed reaction is consistently about 10 g greater than the theoretical weight loss computed from the mass of NaPerc placed in the device. This excess loss of mass is due to the transfer of water vapor in the exiting oxygen stream. Using these values, the average relative humidity of the oxygen produced was found to be about 10%. The mass of oxygen produced at a given moment in time can be converted, using the Ideal Gas Law, to the equivalent volume of oxygen produced at standard temperature and pressure (273.15K, 1 atm.). A graph of oxygen volume vs. time can be differentiated numerically to obtain the rate of oxygen production (L/min) at each time. The rate of oxygen production (Q, L/min) vs. time is shown in FIG. 4.

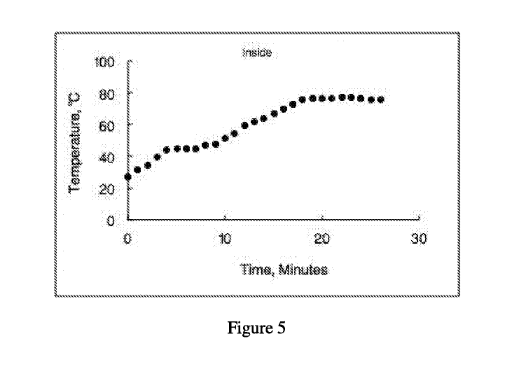

The substantially linear graph of fractional conversion vs. time (FIG. 3), when converted to volumetric flow rate vs. time, exhibited flow rates which rose quickly to 6 L/min and remained above 6 L/min for 18 minutes before falling slowly as the NaPerc is consumed. During the period of the reaction, the temperature inside the reaction chamber was monitored and the plot of reaction temperature vs. time is shown in FIG. 5.

The adiabatic reaction vessel allowed the temperature to be ramped with time. The increase in temperature resulted in an increase in reaction rate and, for the ramp shown in FIG. 5, the increase approximately offset the loss in rate due to the decline in NaPerc (and therefore H₂O₂) availability. As a result, the overall rate of oxygen produced is substantially constant and remained at 6 L/min or more for 18 minutes or longer. The temperature of the reaction mass remained at 80° C. or lower and never approached the boiling point of water. The exiting oxygen cooled quickly as it traveled through the vapor space of the device and in the delivery tubing to the user. After exiting from two feet of silicone tubing, the gas was at room temperature. Importantly, the control of the oxygen producing reaction did not depend on the transfer of heat from the adiabatic reaction chamber to the surroundings and, for this reason, the oxygen delivery rate will not vary when the location of the device is changed.

Thus, Example 3 demonstrates the determination of the amount and rate of oxygen produced by an oxygen generating composition of the disclosure.

### Example 4

A solution was prepared from 0.76 g of FeSO₄, 2.78 g NH₄SCN, and 1.1 g concentrated H₂SO₄. This solution was used in the well-known colorimetric method for determination of peroxides by formation of ferric thiocyanate, a blood red complex formed in solution between ferric iron (Fe³⁺) and thiocyanate ions (SCN⁻). Specifically, the ferrous ion (Fe2+) from ferrous sulfate is readily oxidized to ferric iron by hydrogen peroxide. Therefore, if a gas stream containing hydrogen peroxide is bubble through the solution, the solution will turn to a blood red color. Even tiny amounts of ferric thiocyanate can be detected if the absorbance of the solution is measured on a colorimeter at a wavelength of 450 nm.

Three tests were conducted whereby all of the oxygen produced by the device (˜114 L @STP) was bubbled through 50 mL of the test solution. At the end of each test, the absorbance of the test solution at 450 nm was measured against an unexposed sample of the solution. Under the test conditions, the colorimetric method is capable of detecting as little as 0.5 ppm of H₂O₂ in the oxygen, well below the short term exposure limit (STEL) of 2-3 ppm. The presence of unreacted hydrogen peroxide was not detected by the colorimetric method in any of the experiments.

Thus, Example 4 demonstrates the determination of the amount hydrogen peroxide vapor contained in the oxygen stream produced by an oxygen generating composition of the disclosure.

1. An oxygen generating composition comprising sodium percarbonate;
manganese dioxide; and
a cooling agent selected from the group consisting of trisodium phosphate dodecahydrate, disodium phosphate heptahydrate, disodium phosphate dodecahydrate, and combinations thereof.

2. The composition of claim 1, wherein the composition is substantially free of polyethylene glycol.

3. The composition of claim 1, wherein the composition is a powder mixture.

4. The composition of claim 1, wherein the cooling agent comprises trisodium dodecahydrate.

5. The composition of claim 1, wherein the cooling agent is a powder, a tablet, or a combination thereof.

6. The composition of claim 1, wherein the sodium percarbonate is present in the composition in an amount in a range of about 10 wt % to about 80 wt %.

7. The composition of claim 1, wherein the manganese dioxide is present in the composition in an amount in a range of about 0.001 wt % to about 5.0 wt %, based on the weight of NaPerc.

8. The composition of claim 1, wherein the cooling agent is present in the composition in an amount in a range of about 20 wt % to about 90 wt %.

9. The composition of claim 1, wherein the sodium percarbonate is uncoated and substantially free of foam-producing additives.

10. The composition of claim 1, consisting essentially of
sodium percarbonate;
manganese dioxide; and
trisodium phosphate dodecahydrate.

11. The composition of claim 1, wherein the composition, when stored at a temperature of about 50° C. for at least 1 year, retains at least 99% of the initial amount of sodium percarbonate.

12. The composition of claim 1, further comprising water.

13. The composition of claim 12, wherein the composition has a water to sodium percarbonate ratio in a range of about 1.5:1 to about 2.5:1, or about 1.7:1.

14. The composition of claim 12, wherein the water is substantially free of polyethylene glycol.

15. A method of generating oxygen comprising
contacting the composition of claim 1 with water to generate oxygen.

16. The method of claim 15, wherein the oxygen is generated at a rate of at least 1 L/min, at least 2 L/min, at least 3 L/min, at least 4 L/min, or at least 6 L/min.

17. The method of claim 15, wherein the composition comprises 70 wt % to 75 wt % sodium percarbonate, 0.1 wt % to 0.3 wt % manganese dioxide, and 20 wt % to 30 wt % trisodium phosphate dodecahydrate and oxygen is generated at a rate of at least 6 L/min for 20 minutes.

18. The method of claim 15, wherein the oxygen generation does not produce foam.

19. The method of claim 15, wherein the generation of oxygen is adiabatic.

20. A portable chemical oxygen generator comprising a housing enclosing a reaction chamber comprising a first compartment containing the composition of claim 1 and a second sealed compartment containing water; and
an activation means attached to the housing for opening the sealed second compartment and for contacting the composition with the water.

21. A portable chemical oxygen generator comprising
a housing enclosing a reaction chamber comprising a first compartment containing the composition of claim 1; and
an activation means attached to the housing for providing water and contacting the composition with the water.

22. The portable chemical oxygen generator of claim 20, wherein the water is substantially free of polyethylene glycol.

23. The portable chemical oxygen generator of claim 22, wherein each of the composition and water is substantially free of polyethylene glycol.
